(19) United States
(12) Patent Application Publication
(10) Pub. No.: US 2005/0267423 A1
(43) Pub. Date: Dec. 1, 2005

(54) Ophthalmic surgery preparation system and method

(76) Inventors: Russ Johnson, Spring Grove, IL (US); Mahmood Mohiuddin, Hawthorn Woods, IL (US); Elizabeth Hutson, Mundelein, IL (US); Kelly Williams, Riverside, CA (US); Varsha Kalyankar, Round Lake Beach, IL (US)

Correspondence Address: Kimberly Luna;Paralegal, Allegiance Corporation, 1430 Waukegan Road, McGaw Park, IL, 60085, US

(21) Appl. No.: 11/201,447

(22) Filed: Aug. 10, 2005

Related U.S. Application Data
(63) Continuation-in-part of application No. 10/855,283, filed on May 27, 2004.

(51) Int. Cl. A61M 35/00

(52) U.S. Cl. 604/295; 53/425

(57) ABSTRACT

The invention described herein is a sterile, disposable ophthalmic surgery preparation system and technique that can be introduced directly into the sterile field of surgical preparation without significantly compromising sterile conditions. Both the antimicrobial solution and the container holding the solution can be presented into the sterile field in sterile condition. The antimicrobial solution and container are dimensioned and proportioned for single patient or single procedure usage. The invention includes a sterile, disposable ophthalmic surgery preparation system comprising: a sealed, rupturable flexible container structured as a dropper; the container being filled with a liquid antimicrobial composition formulated for ophthalmic application; wherein the container and composition are proportioned for single application use. The invention further provides a surgical kit and method of preparing a patient for ophthalmic surgery.

### RELATED APPLICATIONS

This application is a continuation-in-part of U.S. patent application Ser. No. 10/855,283 filed May 27, 2004, now pending.

### FIELD OF THE INVENTION

The invention relates to the field of medical devices used in surgery. In particular, the invention pertains to a surgical preparation system for ophthalmic surgery.

### BACKGROUND OF THE INVENTION

In preparation for certain medical and surgical procedures to be performed on the eye or ophthalmic region of the patient, it is desirable to create and maintain a sterile field surrounding the treatment site. This sterile field not only involves the sterile condition of the instrumentation and equipment to be used to carry out such a procedure, but antimicrobial treatment of the tissue site as well.

Ophthalmic antimicrobial solutions that are applied to the ocular region in preparation for surgery are known. Typically, these solutions are applied to the patient from a pre-filled bottle or container containing a larger volume of solution to be used among numerous patients over extended periods of time. The container itself is often stored in non-sterile conditions. Furthermore, the solution container must be handled appropriately at the time of solution application or else it can introduce risk of contamination to the patient.

Povidone iodine, also known as PVP-I or polyvinylpyrrolidone-iodine complex, is a well-known topical antimicrobial agent. Ophthalmic solutions for various eye treatments containing povidone iodine as the active antimicrobial ingredient are also known. Difficulties have been associated, however, with various types of containment and packaging of iodine-containing ophthalmic solutions. For example, problems with iodine leaching, container material and povidone-iodine solution shelf-life have been observed, such as those described in Jauw U.S. Pat. No. 5,178,853 and Bhagwat et al. U.S. Pat. No. 5,126,127. Again, these containers are used in long-term storage and repeated usage context. Thus, they are also susceptible to the problems associated with chemical ingress and contamination, in addition to the contamination risks of a non-sterile container.

The advantages of fluorination of plastic containers have long been exploited in the manufacturing industry as substitutes for breakable glass containers. Examples of chemicals and products that have been contained in fluorinated bottles include acetone, auto additives, lighter fluid, degreasers, electronics chemicals, health and beauty care products, insecticides, kerosene, lubricants, paint thinners, plant growth chemicals, waxes, cleaners and polishes, weed killers and herbicides, wood preservatives, and the like. Joffre U.S. Pat. No. 2,811,468, for example, describes the fluorination of polyethylene containers to render the containers impermeable to atmospheric contamination of their liquid contents.

In the medical field, ethylene oxide sterilization, also referred to as EtO sterilization, is often used in the manufacture and sterilization of sterile medical devices and instruments. Ethylene oxide vaporizes at relatively low temperatures comparable to room temperature. As a result of the substantial chemical activity of the molecules, ethylene oxide gas readily combines with microbial cells to inactivate or kill them. Further, the small size of the ethylene oxide molecule allows penetration into minute openings and porous substances thereby allowing the passage of the sterilant into areas that otherwise, using other sterilization methods, would not have received exposure and antimicrobial effect.

One problem associated with filled container ethylene oxide sterilization processes is the adverse interaction between ethylene oxide and iodine-based antimicrobial solutions. Ethylene oxide gas can readily penetrate thin layers of most plastics. Once inside, the gas is difficult to dissipate quickly. The interaction of ethylene oxide gas and iodine-containing solutions is that it can result in the formation of additional toxic residues. These contaminants include ethylene oxide (EtO), ethylene chlorohydrin (ECH), and ethylene glycol (EG). Iodine-containing antimicrobial liquids are especially sensitive to compromise of their chemical integrity by the ethylene oxide sterilization process. Furthermore, iodo-ethanol contaminants produced by the interaction can cause topical allergic reactions in some patients. In order to provide product safety, residual levels of these contaminants must be at acceptable levels under given circumstances and context (e.g., anticipated product use, location and manner of use, and duration of use).

Excessive or unacceptable levels of ethylene oxide residues in topical applications can cause contact dermatitis, rashes and lesions. Prolonged exposure to ethylene oxide residues have demonstrated carcinogenicity and can be mutagenicity as well.

The problems associated with ethylene oxide contamination would be especially critical with solutions applied to the sensitive tissue of the ophthalmic region. Even further, the small, single application containers would be especially vulnerable to ethylene oxide effect on small contained volumes of liquid therein.

Iodine-based skin antiseptic composition dispenser systems resistant to ethylene oxide gas permeation from sterilization processing are also known, such as that described in Davis et al. U.S. Publ. Appl. No. 2004/0068218 published Apr. 8, 2004. Such dispensing systems, however, involve the use of thickened, multilayered plastic container materials to create the sterilization gas barrier. Further, such systems are not designed for ophthalmic application.

There is a need in the ophthalmic field for ophthalmic preparation systems and methods that reduce the likelihood of compromising sterility during the procedure and can participate in the sterile field. There is also a need for containment of ophthalmic antimicrobial preparation solutions, such as those containing povidone iodine, that are suitable for direct introduction into the sterile field, do not require long-term storage, are not re-used or shared among multiple patients, and which are disposable and easy to manufacture.

### SUMMARY OF THE INVENTION

The invention provides a sterile, disposable ophthalmic surgery preparation system and technique that can be introduced directly into the sterile field of surgical preparation without significantly compromising sterile conditions. It has been discovered that an ophthalmic surgical preparation system, kit and corresponding method can be developed that participate in sterile environment, rather than jeopardize it. According to the invention, both the antimicrobial solution and the container holding the solution are presented into the sterile field in sterile condition. It has been further discovered that an ophthalmic preparation system can be structured in disposable, single use design. Thus, the system of the invention (antimicrobial solution and applicator) are dimensioned and apportioned for single patient or single procedure usage. That is, the invention avoids the repeated handling and storage conditions associated with relatively larger containers wherein the solution is continuously withdrawn for multiple patients. Thus, risks of cross-patient contamination through the solution container and applicator are eliminated. As a further advantage, long-term shelf-life and storage concerns of the solution, especially iodine-containing solutions, are significantly reduced. The sterility of the system can also be maintained through easy-to-use, cost effective, convenient packaging.

The invention provides a sterile, disposable ophthalmic surgery preparation system comprising: a sealed, rupturable flexible container structured as a dropper; the container being filled with a liquid antimicrobial composition formulated for ophthalmic application; wherein the container and composition are proportioned for single application use.

The invention further provides a surgical kit comprising: a sterile, disposable ophthalmic surgery preparation system comprising: a sealed, rupturable flexible container structured as a dropper; the container being filled with a liquid antimicrobial composition formulated for ophthalmic application; wherein the container and composition are proportioned for single application use; and a tray structured to receive the filled container of said system.

The invention also provides a method of preparing a patient for ophthalmic surgery comprising the steps of: a) presenting directly onto a sterile field, a sterile, disposable ophthalmic surgery preparation system comprising: a sealed, rupturable flexible container structured as a dropper; said container being filled with a liquid antimicrobial composition formulated for ophthalmic application; wherein the container and composition are proportioned for single application use; b) rupturing the filled container; and c) applying the liquid antimicrobial composition to an ophthalmic surgery site on the patient.

Furthermore, the invention provides an isotonically-balanced ophthalmic surgical preparation solution formulated for application to the ophthalmic region. In a preferred embodiment, the ophthalmic solution comprises a buffered iodophore or polyvinylpyrrolidone-iodine as an antimicrobial ingredient.

### BRIEF DESCRIPTION OF THE DRAWINGS

The following figures further illustrate the invention, and are not to be construed as necessarily limiting the invention:

FIG. 1 is a top plan view of an ophthalmic preparation system according to one embodiment of the invention.

FIG. 2 is a side view of an ophthalmic preparation system according to one embodiment of the invention.

FIG. 3 is an angled perspective view of an ophthalmic preparation kit showing separation of components according to one embodiment of the invention.

FIG. 4 is a top plan view of the tray portion of an ophthalmic preparation kit according to one embodiment of the invention.

### DETAILED DESCRIPTION OF THE INVENTION

As used herein, the term “surgery” in the context of using the invention is meant to include any invasive or noninvasive medical treatment on or near a patient's ophthalmic region where preliminary antimicrobial treatment of the site would be advantageous.

As used herein, the phrase “isotonically-balanced”, when used in association with the ophthalmic composition of the invention, is meant to refer to the chemical property wherein the composition exhibits a substantially similar osmotic equilibrium to that of 0.9% physiological saline (i.e., extracellular fluid associated with ophthalmic tissue) which produces an equilibrium between osmosis into the cell and out of the cell.

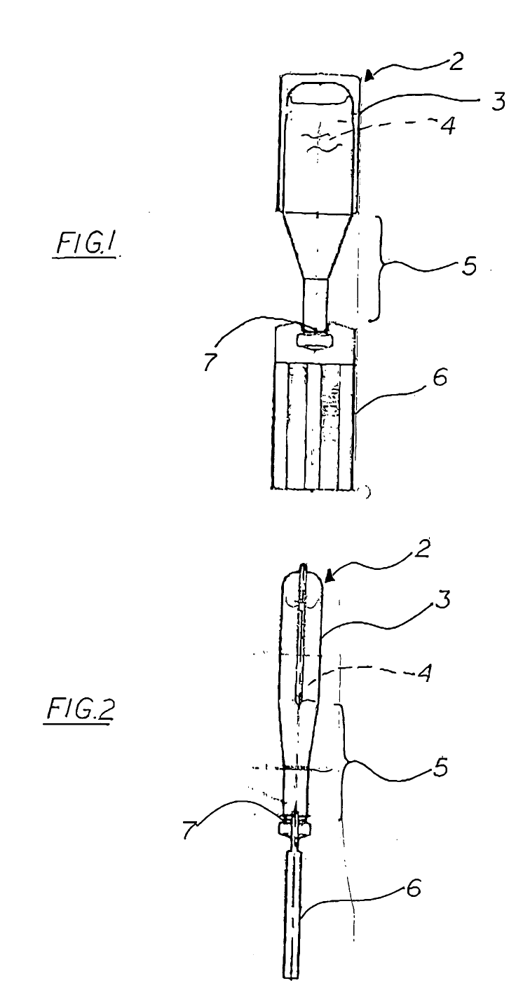

In general and as shown in FIGS. 1 and 2, the invention includes a sterile, disposable ophthalmic surgery preparation system 2 comprising: a sealed, rupturable flexible container 3 structured as a dropper; the container being filled with a liquid antimicrobial composition 4 formulated for ophthalmic application; wherein the container 3 and composition 4 are proportioned for single application use. Single use proportion is an important aspect of the invention, since it substantially reduces the disadvantages associated with long term storage and repeated use of a single container. In general, the container and liquid antimicrobial composition volume of a single system unit ranges from about 0.1 ml to about 3.0 ml in volume, and can be approximately 1.6 ml. Variations in container size and volume are possible depending upon the nature or requirements of the intended surgical procedure.

Preferred containers are those structured with an overall bottle shape (as shown in the figures) with a narrow neck 5 and weakened region 7 that permits rupture, breaking and/or separation of an end portion 6 from the remainder of the bottle-shaped container 3 to create a narrow open tip. Further preferred are containers that are entirely, contiguously and uniformly composed of a single flexible plastic that permits squeezing to effect dispensing of the liquid solution through the open tip. In one embodiment, the container material can be composed of LDPE (low-density polyethylene), or alternatively, a LDPE/HDPE (high-density polyethylene) 50/50 blend. The container itself can be manufactured by conventional techniques and equipment, such as blow-molding techniques (e.g., blow-fill-seal techniques).

Even more preferred are containers and container materials that have been treated or coated to prevent ingress or leaching of either the solution ingredients into the material, and/or external environment gasses into the material and solution. For example, fluorination or aluminum oxide coatings can be applied to the formed container prior to filling with the antimicrobial solution and sealing. An important aspect of the invention is the combination of the container material, the liquid antimicrobial ophthalmic composition, and the ethylene oxide (EtO) sterilization technique. More specifically, the system of the invention retains the desired flexibility of the container material itself, such as that associated with LDPE, to function as a dropper in use, while at the same time affording a barrier against EtO ingress or permeation through the material.

Fluorinated pre-filled containers can be prepared using the process described in parent U.S. patent application Ser. No. 10/855,283 filed May 27, 2004, now pending. In general, one process that can be used can comprise: a) providing a formed empty container; b) subjecting said container to fluorination; c) filling said fluorinated container with liquid antimicrobial solution; d) sealing the filled container; and e) subjecting said filled container to ethylene oxide sterilization. There are two general procedures for fluorination of containers: in-line fluorination and post-molding fluorination. In-line fluorination is described in U.S. Pat. No. 3,862,242 and also known as the AIROPAK® System and incorporated herein by reference. This system utilizes standard blow-molding technology to produce an effective permeation barrier at the inner walls of the container as part of the blow-mold cycle. Post-molding fluorination techniques, such as the commercially available Fluoro-Seal™ process, introduces fluorine gas to containers in a process reactor.

The fluorinated container can subsequently filled with liquid antimicrobial ophthalmic solution and hermetically sealed in accordance with readily available techniques, equipment and materials. The filled and sealed containers with liquid antimicrobial ophthalmic solution within, can then be subjected to the sterilization process.

In an alternative embodiment, the container can be initially be filled with liquid antimicrobial ophthalmic solution, hermetically sealed, and then the exterior of the filled and sealed container can be subsequently subjected to the fluorination process. Again, the filled and sealed fluorinated container can then be further subjected to the ethylene oxide sterilization process to prepare the completed product.

In general, ethylene oxide (EtO) sterilization process involves four basic phases: 1) air removal; 2) steam injection and conditioning dwell; 3) EtO injection and gas dwell; and 4) gas purge and air inbleed. The parameters for the sterilization step include temperature, pressure, humidity, ethylene oxide concentration, and gas dwell time. The sterilization process must be effected to a degree that ensures a 10⁻⁶ sterility assurance level (SAL) without causing deleterious effects to the product or packaging. The process parameters can be adjusted in order to optimize fluorination effect (where fluorination coating technique is used) and results. Controlled and effective EtO sterilization processes account for the medical product being sterilized, e.g., antimicrobial liquid solution, and the permeability of its packaging, e.g., bottle or container, and also produce complete and consistent results.

The advantage of such treated containers is that they are resistant to ethylene oxide ingress, thereby reducing the undesirable creation of contaminants, such as ethylene chlorhexidine. As a further advantage, fluorination coating or aluminum oxide coating can avoid the need for thickened container walls thereby maintaining the material flexibility, and/or multilayered container materials utilizing different materials or plastics which are more complicated to manufacture.

The system of the invention includes a liquid antimicrobial composition formulated for ophthalmic application. More specifically, antimicrobial formulations for use in ophthalmic surgery should at the same time accommodate the sensitive nature of the surrounding tissue region associated with the surgical site, i.e., the ophthalmic tissue region. In a preferred embodiment, therefore, the ophthalmic solution comprises a buffered iodophore or polyvinylpyrrolidone-iodine complex as an antimicrobial ingredient.

The following is an example of one buffered povidone iodine-containing surgical preparation formulation that can be used in conjunction with the invention.

### EXAMPLES

### Example 1

### Preparation of 5.0% PVP-I Eye Surgical Preparation Solution

An adjusted amount of USP grade povidone-iodine (PVP-I) powder is gradually added to 80.00% w/w of the total formulation weight of USP sterile water for inhalation in a 900 ml beaker. The total amount of PVP-I to be added must be measured so that it complies with the relevant USP guidelines for percent available iodine. The solution is mixed using an IKA Labortechnik™ RW16 basic mixer until all the PVP-I powder has been dissolved. Polyethylene glycol nonylphenyl ether (e.g., Igepal™ CO-630 available from Rhodia, Cranbury, N.J.) and glycerin are each gradually added into the solution under mixing conditions until homogeneity is reached.

The buffer system is prepared separately. In a 100 ml beaker, 0.15% w/w of citric acid, 0.15% w/w of sodium hydroxide, and 0.12% w/w of anhydrous sodium phosphate dibasic are added to 14.08% w/w USP grade sterile water for inhalation. The buffer system is then mixed under stirring conditions using a stir bar and stirring plate until a uniform, clear solution results. The total amount of the buffer solution is then added into the PVP-I-containing solution into a 900 ml beaker, producing a pH of about 6.1.

The total volume of 5% PVP-I is determined. Then, the amount of sodium chloride to be added to the solution is calculated by multiplying 5.99 g/l by the total volume (in liters) of the solution. The calculated amount of sodium chloride is weighed and then added to the final solution and mixed until dissolved.

The ingredients and amounts of a preferred formulation that can be prepared using the above example are set forth in the following table:

TABLE 1
5% PVP-I Surgical Eye Preparation Composition

| Ingredient: | Amount (w/w %) |
|---|---|
| PVP-I (polyvinylpyrrolidone-iodine | 5.00 |
| complex) USP | |
| Water USP WFI | 94.08 |
| Glycerin | 0.25 |
| Igepal ™ CO-630 (polyethylene | 0.25 |
| glycol nonylphenyl ether) | |
| Citric acid | 0.15 |
| Sodium hydroxide | 0.15 |
| Anhydrous sodium phosphate dibasic | 0.12 |
| Total: | 100.00 |
| Sodium chloride | 5.99 g/L |

The preferred composition has a pH ranging from about 5.5 to about 6.5, and is isotonically balanced with 0.9% sterile saline.

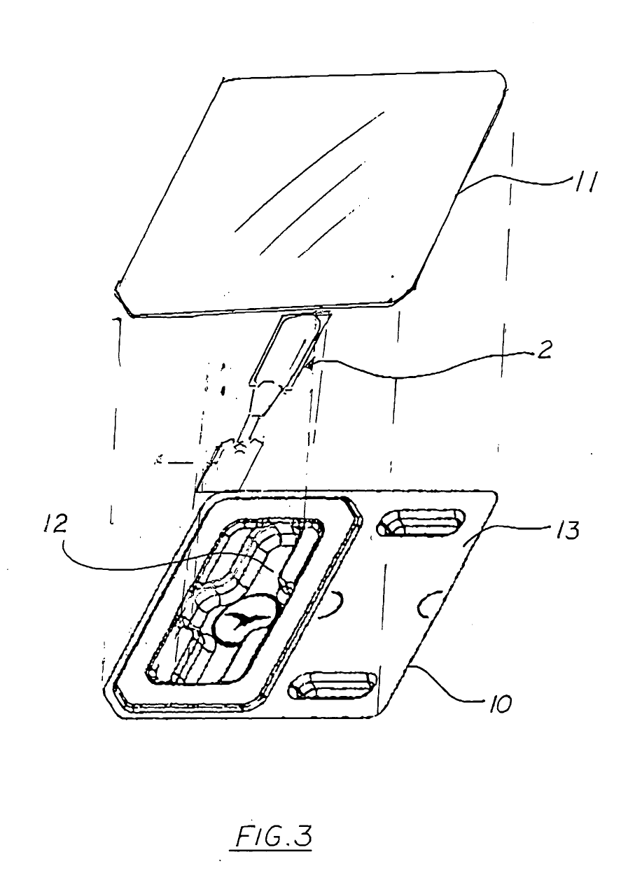

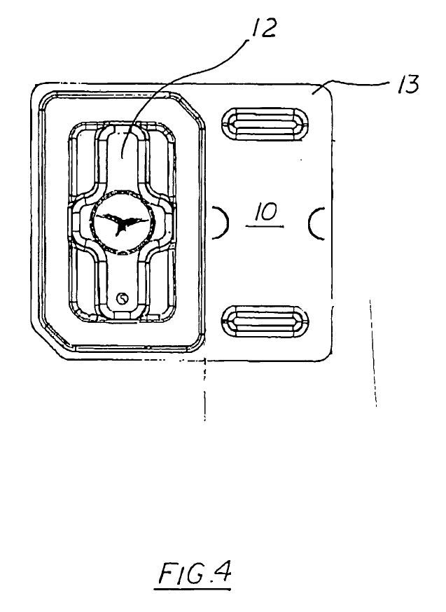

Referring now to FIG. 3, the invention also includes a surgical kit comprising: a sterile, disposable ophthalmic surgery preparation system 2 comprising: a sealed, rupturable flexible container 3 (see FIGS. 1 and 2) structured as a dropper; the container 3 being filled with a liquid antimicrobial composition 4 formulated for ophthalmic application; wherein the container and composition are proportioned for single application use; and a tray 10 (see FIGS. 3 and 4) structured to receive the filled container of the system 2 (as illustrated in FIG. 3).

In one embodiment and as shown in FIGS. 3 and 4, the tray 10 can be formed and structured to have a recess or pocket 12 dimensioned to receive the container, as well as to receive additional components that may be included within the kit. The tray 10 of the kit can be formed from conventional thermoformable plastics readily available to those skilled in the art. Alternatively, the tray 10 can be composed of a metallic material, such as aluminum.

The tray 10 can further comprise a peelable or removable lid 11 that can be sealed onto the tray 10 and circumscribing the perimeter 13 of the tray 10. The lid 11 can be composed of a monolayer or laminated metallic or plastic material. Suitable lid materials include, but are not limited to, Tyvek™. Thus, the contents of the tray are capable of direct and sterile presentation into the operative field once the lid has been removed.

The kit can further comprise secondary components (not shown in the figures). Examples of suitable secondary components that can be included within the kit are selected from the group consisting of blotting towels, gauze, swabs, gloves, sponges, saline solution, wraps, pouches, and combinations thereof.

One of the main advantages of the system of the invention is that it improves patient preparation for ophthalmic surgery by reducing the likelihood of contamination. Thus, the invention includes a method of preparing a patient for ophthalmic surgery comprising the steps of: a) presenting directly onto a sterile field, a sealed, rupturable flexible container structured as a dropper; said container being filled with a liquid antimicrobial composition formulated for ophthalmic application; wherein the container and composition are proportioned for single application use; b) rupturing the filled container; and c) applying the liquid antimicrobial composition to an ophthalmic surgery site on the patient.

For example, the kit is presented to the user and the lid can be peeled back from the tray, thereby exposing the tray contents. The tray can be “flipped” to deposit the ophthalmic surgical preparation system (pre-filled dropper) directly onto the sterile field, or alternatively, the practitioner can remove the system with a gloved hand. The tip of the container can then be twisted-off or otherwise broken and separated from the container. Finally, the container functions as a dropper and can be gently squeezed to dispense the composition into the tissue site. Thus, the invention affords the advantage of its presentability, and its maintainability, as a sterile system during the procedure.

### INDUSTRIAL APPLICABILITY

The invention is useful in the preparation of patients for ophthalmic treatments and surgery. As a result of its sterile and single-use attributes, the ophthalmic surgical preparation system of the invention allows for direct presentation onto the sterile operative field and reduces the likelihood of introducing contaminants onto the field or patient.

The invention has been described herein above with reference to various embodiments and techniques. It will be understood by one of ordinary skill that reasonable variations and modifications of such embodiments and techniques are possible without significantly departing from either the spirit or scope of the invention defined by the claims set forth below.

1. A sterile, disposable ophthalmic surgery preparation system comprising:
a sealed, rupturable flexible container structured as a dropper; said container being filled with a liquid antimicrobial composition formulated for ophthalmic application;
wherein said container and composition are proportioned for single application use.

2. The system according to claim 1, wherein said container has been treated to inhibit ethylene oxide gas ingress into said container.

3. The system according to claim 2, wherein said treatment comprises application of aluminum oxide coating onto said container surface.

4. The system according to claim 2, wherein said treatment comprises fluorination process applied to said container surface.

5. The system according to claim 1, wherein said container is composed of a plastic material comprising low-density polyethylene.

6. The system according to claim 1, wherein said liquid antimicrobial composition comprises iodine.

7. The system according to claim 6, wherein said liquid antimicrobial composition comprises polyvinylpyrrolidone-iodine.

8. The system according to claim 7, wherein said liquid antimicrobial composition comprises up to about 5% by weight polyvinylpyrrolidone-iodine of the total liquid composition.

9. A surgical kit comprising:
a sterile, disposable ophthalmic surgery preparation system comprising:
a sealed, rupturable flexible container structured as a dropper; said container being filled with a liquid antimicrobial composition formulated for ophthalmic application;
wherein said container and composition are proportioned for single application use; and
a tray structured to receive said filled container of said system.

10. The kit according to claim 9, wherein said container has been treated to inhibit ethylene oxide gas ingress into said container.

11. The kit according to claim 10, wherein said treatment comprises application of aluminum oxide coating onto said container surface.

12. The kit according to claim 10, wherein said treatment comprises fluorination process applied to said container surface.

13. The kit according to claim 9, wherein said container is composed of a plastic material comprising low-density polyethylene.

14. The kit according to claim 9, wherein said liquid antimicrobial composition comprises iodine.

15. The kit according to claim 14, wherein said liquid antimicrobial composition comprises buffered polyvinylpyrrolidone-iodine.

16. The kit according to claim 15, wherein said liquid antimicrobial composition comprises up to about 5% by weight polyvinylpyrrolidone-iodine of the total liquid composition.

17. A method of preparing a patient for ophthalmic surgery comprising the steps of:
a) presenting directly onto a sterile field, a sterile, disposable ophthalmic surgery preparation system comprising:
a sealed, rupturable flexible container structured as a dropper; said container being filled with a liquid antimicrobial composition formulated for ophthalmic application;
wherein said container and composition are proportioned for single application use;
b) rupturing said filled container; and
c) applying said liquid antimicrobial composition to an ophthalmic surgery site on said patient.

18. The method according to claim 17, wherein said container has been treated to inhibit ethylene oxide gas ingress into said container.

19. The method according to claim 18, wherein said treatment comprises application of an aluminum oxide coating onto said container surface.

20. The method according to claim 18, wherein said treatment comprises fluorination process applied to said container surface.

21. The method according to claim 17, wherein said container is composed of plastic material comprising low-density polyethylene.

22. The method according to claim 17, wherein said liquid antimicrobial composition comprises iodine.

23. The method according to claim 22, wherein said liquid antimicrobial composition comprises buffered polyvinylpyrrolidone-iodine.

24. The method according to claim 23, wherein said liquid antimicrobial composition comprises up to about 5% by weight polyvinylpyrrolidone-iodine of the total liquid composition.

25. An isotonically-balanced ophthalmic surgical preparation composition comprising:
PVP-I present in an amount of about 5.0% by weight of the total composition;
glycerin present in an amount of about 0.25% by weight of the total composition;
polyethylene glycol nonylphenyl ether present in an amount of about 0.25% by weight of the total composition;
a buffer system consisting essentially of:
citric acid present in an amount of about 0.15% by weight of the total composition;
sodium hydroxide present in an amount of about 0.15% by weight of the total composition;
dibasic sodium phosphate present in an amount of about 0.12% by weight of the total composition; and
sodium chloride present in an amount of about 5.99 g/l;
wherein said composition has a pH ranging from about 5.0 to about 6.5; and
wherein said composition is isotonically balanced with 0.9% sterile saline.
